(19) United States
(12) Patent Application Publication
(10) Pub. No.: US 2026/0123738 A1
(43) Pub. Date: May 7, 2026

(54) FACE COMPRESSION BEAUTY MASK

(71) Applicant: Kyung Yong KOH, Seoul (KR)

(72) Inventor: Kyung Yong KOH, Seoul (KR)

(21) Appl. No.: 19/117,734

(22) PCT Filed: Sep. 19, 2023
(86) PCT No.: PCT/KR2023/014138

§ 371 Date: Apr. 2, 2025

(30) Foreign Application Priority Data
Oct. 21, 2022 (KR) 10-2022-0136598

(51) Int. Cl. A45D 44/22; A61H 23/00; A61N 1/04; A61N 1/32; A61N 1/36

(52) U.S. Cl. CPC A45D 44/22; A61H 23/00; A61N 1/0472; A61N 1/328; A61N 1/36014; A45D 2200/207; A61H 2201/10; A61H 2201/1607; A61H 2201/165; A61H 2205/022

(57) ABSTRACT

A face compression beauty mask according to an embodiment of the present invention comprises: a first mask 10, which generates facial surface data by three-dimensionally scanning the face of a user, has a contact surface 12 formed on the basis of the facial surface data, and is formed of a soft material; a second mask 20, which is adhered to the outer surface of the first mask 10 and is provided as a durable material; side bands 30, which are fastened to both side surfaces of the second mask 20 so as to be arranged toward the back of the head of the user, and have elasticity; an upper band 40, which is fastened to the upper side of the second mask 20 so as to be arranged toward the back of the head of the user by passing over the top of the head of the user, and has elasticity; and a buckle unit 50 for fastening both side bands 30 to the upper band 40 at the back of the head of the user.

### TECHNICAL FIELD

The present invention relates to a face compression beauty mask that can improve the appearance of aging on the face.

### BACKGROUND ART

Generally, as people age, their skin can sag due to the influence of gravity, and depending on the degree of sagging skin, the face may look older.

Conventionally, efforts were made to provide nutrients to the skin to make the face look a little younger, but the results of the efforts were still insufficient and unsatisfactory.

In addition, some people can receive skin care at a dermatologist, but there is the issue of high cost and the discomfort of having to visit a hospital to receive skin care.

### PRIOR ART LITERATURE

### Patent Literature

(Patent Literature 1) Korean Unexamined Patent Publication no. KR 10-2020-0087461 A
(Patent Literature 2) Korean Unexamined Patent Publication no. KR10-2017-0049888 A

### DETAILED DESCRIPTION OF THE INVENTION

### Technical Problem

The present invention has been proposed to improve the above-mentioned problems in the prior arts, and an object thereof is to provide a face compression beauty mask that allows for easy skin care at low cost.

Another object of the present invention is to provide a face compression beauty mask that allows force to act on a facial skin so as to pull up the facial skin toward the forehead.

Another object of the present invention is to provide a face compression beauty mask that compresses the entire face except for the eyes, nostrils and mouth, allows the compressed area to be protrusively formed into countless small hexagonal shapes, which are the most stable in nature, and pulls up the skin sagging with the advance of age to make it look slimmer.

Yet another object of the present invention is to provide a face compression beauty mask that can perform beauty care without spending expensive costs for beauty care such as face lift surgery.

A further object of the present invention is to provide a face compression beauty mask that re-models the face and hair style of managed persons every 2 to 5 years and modifies the mask to fit the deformed face, thereby allowing for wrinkle care.

### Technical Solution

In order to achieve the above technical objects, according to certain aspects of the present invention, there is provided a face compression beauty mask comprising: a first mask 10, which generates facial surface data by three-dimensionally scanning the face of a user, has a contact surface 12 formed on the basis of the facial surface data, and is formed of a soft material; a second mask 20, which is adhered to the outer surface of the first mask 10 and is provided as a durable material; side bands 30, which are fastened to both side surfaces of the second mask 20 so as to be arranged toward the back of the head of the user, and have elasticity; an upper band 40, which is fastened to the upper side of the second mask 20 so as to be arranged toward the back of the head of the user by passing over the top of the head of the user, and has elasticity; and a buckle unit 50 for fastening both side bands 30 to the upper band 40 at the back of the head of the user.

In the face compression beauty mask according to certain embodiments of the present invention, the upper band 40 may have stronger elasticity than the side band 30.

In the face compression beauty mask according to certain embodiments of the present invention, the first mask 10 may include a mask in which polygonal spaces are densely formed on the contact surface 12, one space is from 0.2 mm to 1 mm in size, and the width between the space and the adjacent space is from 0.2 mm to 1 mm in size.

In the face compression beauty mask according to certain embodiments of the present invention, the material of the first mask 10 may be silicone, and the material of the second mask 20 may be a synthetic resin or a metal.

In the face compression beauty mask according to certain embodiments of the present invention, a first micro hole 16 is formed on the first mask 10, and a second micro hole 26 is formed on the second mask 20, thereby allowing air to pass through the first micro hole 16 and the second micro hole 26.

In the face compression beauty mask according to certain embodiments of the present invention, an electrode 18 is formed on the first mask 10, and a fine current is provided to the electrode 18, thereby allowing the fine current to flow to the face of the user.

In the face compression beauty mask according to certain embodiments of the present invention, a vibrator 60 that generates vibration is further disposed on one side of the second mask 20 to apply fine vibrations to the facial skin of the user while the user is undergoing skin beauty care.

Specific details of other embodiments are included in the detailed description and accompanying drawings.

### Advantageous Effects

The face compression beauty mask according to certain embodiments of the present invention has the advantage that the facial skin can be easily managed at home without visiting a dermatologist, thereby reducing the cost of skin care.

The face compression beauty mask according to certain embodiments of the present invention exerts force to pull up the facial skin toward the forehead in an compression state when worn on the face, thereby expecting the effect of delaying sagging of the facial skin and delaying the transformation into presbyopia.

The face compression beauty mask according to certain embodiments of the present invention compresses the entire face except for the eyes, nostrils and mouth, and allows the compressed area to be protrusively formed into countless small hexagonal shapes, which are the most stable in nature, which can expect the effect of pressing the skin sagging with the advance of age and fixing the skin, and further can prevent the formation of wrinkles, and make the face of managed persons look slimmer.

The face compression beauty mask according to certain embodiments of the present invention can perform beauty care without spending expensive costs for beauty care such as face lift surgery.

The face compression beauty mask according to certain embodiments of the present invention can remodel the face and hair style of managed persons every 2 to 5 years and modify the mask to fit the deformed face, allowing for wrinkle care.

The face compression beauty mask according to certain embodiments of the present invention can form fine holes densely from the outer surface to the inner surface, thereby allowing air to pass through, and reducing the stuffiness while it is receiving the care.

### BRIEF DESCRIPTION OF THE DRAWINGS

FIG. 1 is a flow chart for explaining the process of manufacturing a face compression beauty mask according to an embodiment of the present invention.

FIG. 2 is a drawing showing an example of scanning a face in the process of manufacturing a face compression beauty mask according to an embodiment of the present invention.

FIG. 3 is a diagram for explaining an example of producing a first mask in the process of manufacturing a face compression beauty mask according to an embodiment of the present invention.

FIGS. 4 and 5 are drawings for explaining an example of joining a first mask and a second mask in the process of manufacturing a face compression beauty mask according to an embodiment of the present invention.

FIG. 6 is a diagram for explaining the overall appearance of a face compression beauty mask according to an embodiment of the present invention.

FIG. 7 is a diagram for explaining an example of joining a side band and an upper band in a face compression beauty mask according to an embodiment of the present invention.

※ Description of Symbols

| | |
|---|---|
| 10, 20: first, second mask | 12: contact surface |
| 14, 24: first, second breathing hole | |
| 30: side band | 32: side buckle |
| 40: upper band | 42: upper buckle |
| 50: buckle unit | 100: scanner |

### DETAILED DESCRIPTION OF THE EMBODIMENTS

The advantages and features of the present invention, as well as the method for achieving them, will become apparent by describing in detail the embodiments of the present disclosure with reference to the accompanying drawings.

Hereinafter, specific embodiments of the present invention will be described in detail with reference to the accompanying drawings. It should be understood that the embodiments disclosed herein are illustrative only for better understanding of the present invention, and that the present disclosure may be modified in various ways, differently from the embodiments described herein. However, in case it is determined that in describing embodiments, detailed explanation of the functions and configurations of related known technologies may unnecessarily confuse the gist of the disclosure, the detailed explanation will be omitted. In addition, for ease understanding of the present disclosure, the accompanying drawings are not drawn to real scale, but the dimensions of some components may be exaggerated.

Meanwhile, terms such as “first,” “second” and the like may be used to describe various elements, but the terms are not intended to limit the elements. Such terms are used only for the purpose of distinguishing one element from another element. For example, the first element may be named the second element, and similarly, the second element may also be named the first element, without departing from the scope of the present invention.

On the other hand, the terms described below are terms set in consideration of their functions in the present invention, and may vary depending on the intention or practice of the manufacturer, so their definitions should be made based on the contents throughout this specification.

Throughout the specification, the same reference numerals refer to the same components.

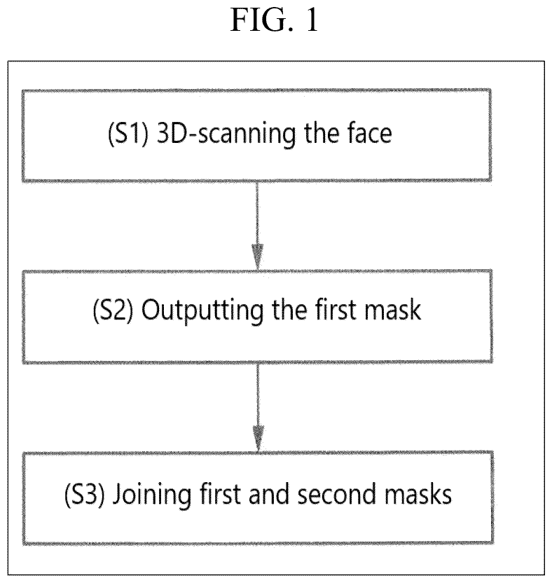

Now, a face compression beauty mask according to an embodiment of the present invention will be described with reference to FIGS. 1 to 7.

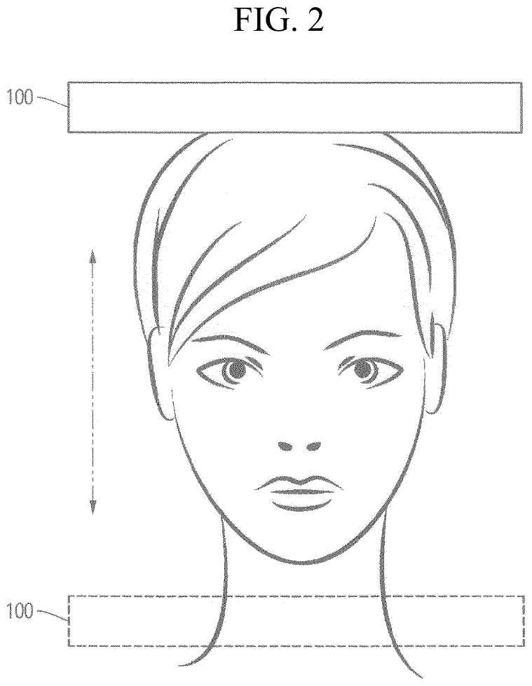

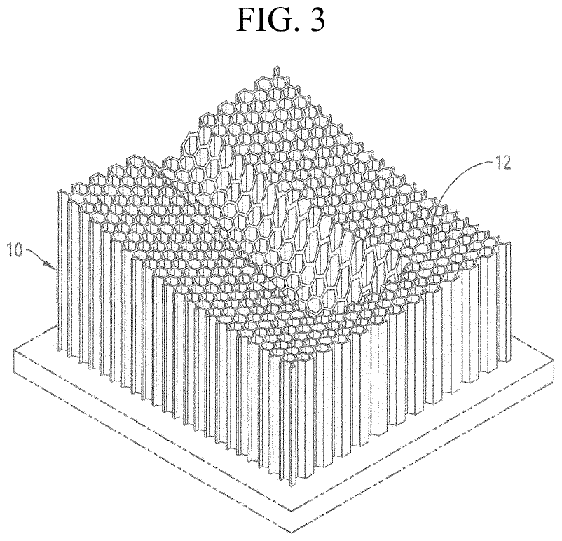

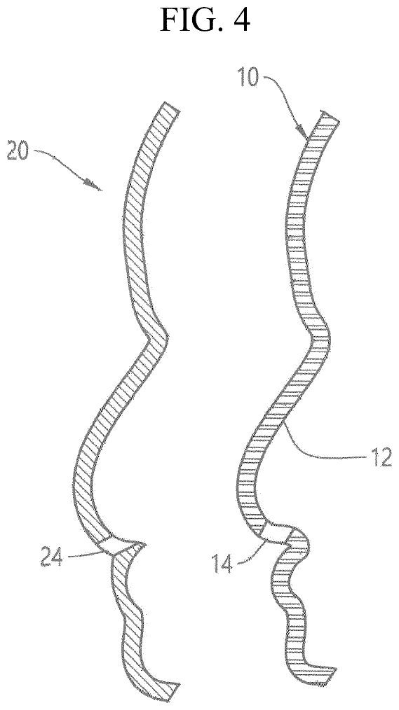

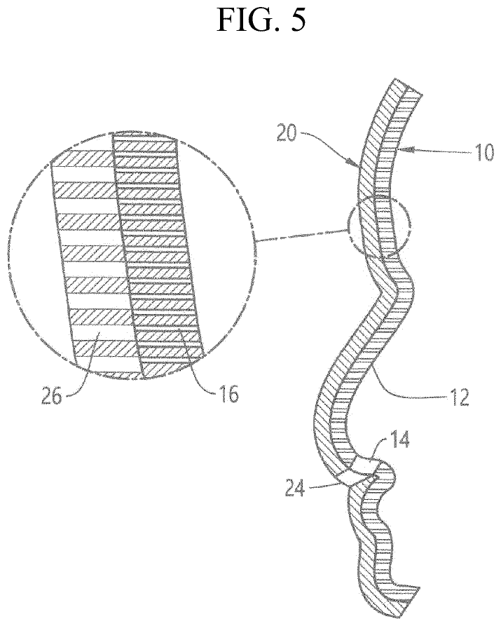

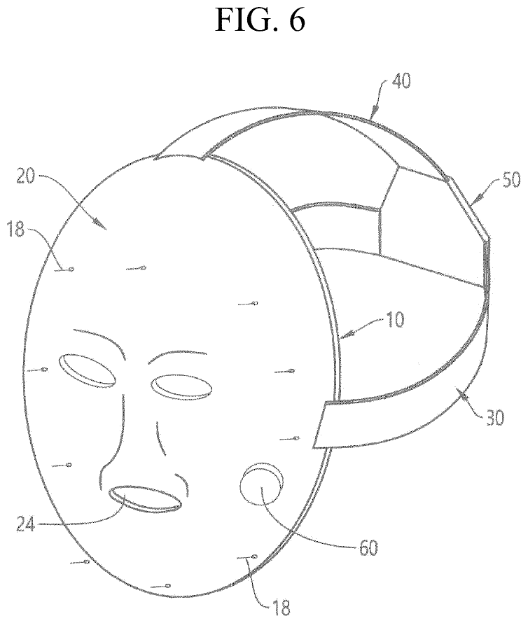

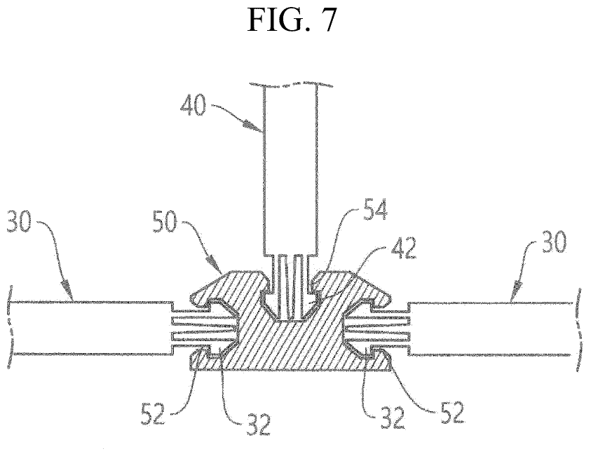

FIG. 1 is a flow chart for explaining the process of manufacturing a face compression beauty mask according to an embodiment of the present invention. FIG. 2 is a drawing showing an example of scanning a face in the process of manufacturing a face compression beauty mask according to an embodiment of the present invention. FIG. 3 is a diagram for explaining an example of producing a first mask in the process of manufacturing a face compression beauty mask according to an embodiment of the present invention. FIGS. 4 and 5 are drawings for explaining an example of joining a first mask and a second mask in the process of manufacturing a face compression beauty mask according to an embodiment of the present invention. FIG. 6 is a diagram for explaining the overall appearance of a face compression beauty mask according to an embodiment of the present invention. FIG. 7 is a diagram for explaining an example of joining a side band and an upper band in a face compression beauty mask according to an embodiment of the present invention.

First, the manufacturing process of the face compression beauty mask according to certain embodiments of the present invention is described with reference to FIG. 1.

Step 1 (S1): As shown in FIG. 2, a scanner 100 may be used to scan the face, thereby generating facial surface data of a user.

Step 2 (S2): A three-dimensional printer may be used to output a first mask 10 as shown in FIG. 3.

The first mask 10 has a contact surface 12, wherein the contact surface 12 is based on the previously generated facial surface data of the user. For example, looking at FIG. 3, it can be seen that the contact surface 12 is formed with a concave nose shape at the position corresponding to the nose.

As a result, since the appearance of the face differs for each user, each specific user can have a unique first mask 10.

Step 3 (S3): The first mask 10 and the second mask 20 can be joined together to complete the cosmetic mask.

As described above, the first mask 10 generates facial surface data by three-dimensionally scanning the face of a user, and has a contact surface 12 formed on the basis of the facial surface data.

The first mask 10 is a portion that touches the face of the user, and thus may be formed of a soft material, so that the user may not feel uncomfortable when wearing the beauty mask.

In addition, the first mask 10 may have a first breathing hole 14 formed in a portion where the nose of a user is located, as shown in FIGS. 4 and 5.

The second mask 20 is attached to the outer surface of the first mask 10, as shown in FIGS. 4 and 5. The second mask 20 is provided as a durable material.

In addition, the second mask 20 may have a second breathing hole 24 formed so as to pass through the first breathing hole 14, as shown in FIGS. 4 and 5.

When the user wears the facial compression beauty mask according to certain embodiments of the present invention, the user can breathe through the first and second breathing holes 14 and 24.

On the other hand, the second mask 20 is a component that can be commonly used by all users, and the outer surface of the first mask 10 can be fabricated in a shape that matches the facial surface of the second mask 20.

The side band 30 can be fastened to both sides of the second mask 20 as shown in FIG. 6 and disposed toward the back of the head of the user. Further, the side band 30 has elasticity.

The upper band 40 can be fastened to the upper side of the second mask 20 as shown in FIG. 6 so as to be arranged toward the back of the head of the user by passing over the top of the head of the user, and has elasticity. Further, the upper band 40 has elasticity.

The buckle unit 50 can be disposed toward the back of the head of the user and fastens the side band 30 and the upper band 40 on both sides.

When a user wears the face compression beauty mask according to an embodiment of the present invention, the side band 30 is worn first, and then the upper band 40 is worn later.

The buckle unit 50 is described in more detail. The buckle unit 50 has a first female fastening hole 52 formed on both sides, and a second female fastening hole 54 formed on the upper side.

A first male fastening hole 32 is formed at the end of the side band 30, and a second male fastening hole 42 is formed at the end of the upper band 40.

The first and second male fastening holes 32 and 42 have a side face shape formed in an arrow shape and have elasticity.

That is, when the first male fastener 32 is mounted on the first female fastener 52, it is easily fitted while being deflated due to its elasticity, and the elastic restoring force acts to spread in the arrow shape, so that the side band 30 can be fastened to the buckle unit 50.

Similarly, when the second male fastener 42 is mounted on the second female fastener 54, it is easily fitted while being deflated due to its elasticity, and the elastic restoring force acts to spread in the arrow shape, so that the upper band 40 can be fastened to the buckle unit 50.

Subsequently, when the user wants to take off the beauty mask, the user can pull out the side buckle 32 and the upper buckle 42 from the buckle unit 50 while applying force to the side buckle 32 and the upper buckle 42, and take it off.

The face compression beauty mask according to certain embodiments of the present invention configured as described above is to wait for a certain period of time in a state of simply wearing on the face, and the method to use it is simple and no additional costs are incurred other than the initial cost of purchasing the beauty mask. That is, the facial skin can be easily managed at home even without visiting a dermatologist, which offers the advantage of almost no skin care costs being incurred.

Further, when the face compression beauty mask according to certain embodiments of the present invention is worn on the face, it exerts force so as to pull up the facial skin toward the forehead due to the elasticity of the upper band 40, under the state of compressing the facial skin, so that the effect of pulling up the facial skin can be expected, and further, the effect of delaying sagging of the skin and deforming into presbyopia can be expected.

Further, the face compression beauty mask according to certain embodiments of the present invention compresses the entire face except for the eyes, nostrils and mouth, and allows the compressed area to be protrusively formed into countless small hexagonal shapes, which are the most stable in nature, which can expect the effect of pressing the skin sagging with the advance of age and fixing the skin, and further can prevent the formation of wrinkles, and make the face of managed persons look slimmer.

Further, the face compression beauty mask according to certain embodiments of the present invention can perform beauty care without spending expensive costs for beauty care such as face lift surgery.

Further, the face compression beauty mask according to certain embodiments of the present invention can remodel the face and hair style of managed persons every 2 to 5 years and modify the mask to fit the deformed face, allowing for wrinkle care.

Further, the face compression beauty mask according to certain embodiments of the present invention can form fine holes densely from the outer surface to the inner surface, thereby allowing air to pass through, and reducing the stuffiness while it is receiving the care.

On the one hand, the face compression beauty mask according to certain embodiments of the present invention has been described as being limited to the face, but the first and second masks 10 and 20 can compress to the lower part of the user's chin or extend up to the user's neck to compress or massage the user's neck.

On the other hand, the upper band 40 may have stronger elasticity than the side band 30.

The side band 30 applies force so that the beauty mask closely adheres to the face.

The upper band 40 applies force so that the beauty mask raises toward the forehead.

That is, under the state where the face compression beauty mask according to certain embodiments of the present invention is in close contact with the face, the upper band 40 has a stronger elasticity, and thus, the beauty mask receives a force that tries to push upward while being in close contact with the facial skin. During this process, it can be expected that the facial skin receives a force in the upward direction, thereby pulling up the skin.

Meanwhile, the face compression beauty mask according to certain embodiments of the present invention can have a polygonal space densely formed on the contact surface 12 of the first mask 10, as shown in FIG. 3.

One space formed on the contact surface 12 can be from 0.2 mm to 1 mm in size, and the width between the space and the adjacent space can be from 0.2 mm to 1 mm in size, so that when the first mask 10 is compressed against the face, the contact surface is limited to the area excluding the space.

If the size of the space is too small, the user may feel stuffy, but if the size of the space is larger than 0.2 mm, the user may feel the contact surface 12 being in close contact with the skin without feeling stuffy, and the contact surface 12 can actually be in close contact with the skin.

On the other hand, if the size of the space is too large, a pressed mark may be formed on the face of the user, and thus it needs to be appropriately limited. If the size of the space is smaller than 1 mm, a pressed mark may not be formed on the skin surface due to the elasticity of the skin, and even if it is formed, it may disappear quickly.

In addition, as previously described, when the space is continuously formed in the up-down and left-right directions on the contact surface 12, the width between one space and the adjacent space may be from 0.2 mm to 1 mm.

Since the pressure is determined by the force applied per unit area, it is preferable to set the width between spaces narrowly in order to increase the pressure acting on the skin. In order to form an appropriate pressure, the width between the spaces may be preferably formed into a size of 0.2 mm to 1 mm.

That is, the surface area where the facial skin comes into contact with the contact surface 12 is narrowly limited, so that the contact pressure when the first mask 10 is pressed against the facial skin can be further increased, and the effect of pulling the facial skin while pressing the skin with high pressure and pulling up it toward the forehead can be expected.

On the other hand, in the face compression beauty mask according to certain embodiments of the present invention, the first mask 10 may be made of silicone, and the second mask 20 may be made of a synthetic resin or a metal.

As a result, the first mask 10 can be stably in close contact with the facial skin without causing damage to the facial skin even when it touches the facial skin, and the user can eliminate any discomfort that may be felt from the hard material.

In addition, the second mask 20 is provided with a hard material, and therefore, when applying pressure using the side band 30 and the upper band 40, the pressure is transmitted to the facial skin, so that pressure can be uniformly formed on the entire facial skin.

In addition, in the face compression beauty mask according to certain embodiments of the present invention, the first micro holes 16 may be densely formed in the first mask 10, and the second micro holes 26 may be densely formed in the second mask 20 as shown in FIG. 5. The positions of the first micro holes 16 and the second micro holes 26 may not exactly match each other, and the size of the second micro holes 26 is formed much larger than the size of the first micro holes 16, so that air can pass through the first micro holes 16 and the second micro holes 26.

That is, the face compression beauty mask according to certain embodiments of the present invention can form micro holes densely from the outer surface to the inner surface, thereby allowing air to pass through and reducing the feeling of stuffiness while receiving care.

Further, the face compression beauty mask according to certain embodiments of the present invention can further enhance the beauty effect when used together with a beauty cream or a nutrition cream.

In addition, as shown in FIG. 6, the face compression beauty mask according to certain embodiments of the present invention can have an electrode 18 formed on the surface of the first mask 10 that touches the face, and can provide fine current through the electrode 18 to provide fine current stimulation to the facial skin, and such fine current stimulation can be helpful for skin beauty care.

The face compression beauty mask according to certain embodiments of the present invention can use fine current when it is determined necessary according to the user's will.

Furthermore, the face compression beauty mask according to certain embodiments of the present invention can have a vibrator 60 on the second mask 20, wherein the vibrator 60 can generate fine vibrations. Thereby, fine vibrations can be transmitted to the facial skin while the user is receiving care in the state of wearing the beauty mask, and the fine vibrations can be helpful for skin beauty care.

The face compression beauty mask according to certain embodiments of the present invention can use fine vibrations when it is determined necessary according to the user's will.

Although the embodiments of the present invention have been described above with reference to the accompanying drawings, those skilled in the art to which the present invention pertains will understand that the present invention can be implemented in other specific forms without changing the technical idea or essential features thereof.

Therefore, the embodiments described above should be understood as illustrative and not restrictive in all respects, and the scope of the present invention is indicated by the claims described below, and all changes or modifications derived from the meaning and scope of the claims and their equivalent concepts should be interpreted as being included in the scope of the present invention.

### INDUSTRIAL APPLICABILITY

The face compression beauty mask according to certain embodiments of the present invention can be used for facial skin care

1. A face compression beauty mask comprising:
a first mask 10, which generates facial surface data by three-dimensionally scanning the face of a user, has a contact surface 12 formed on the basis of the facial surface data, and is formed of a soft material;
a second mask 20, which is adhered to the outer surface of the first mask 10 and is provided as a durable material;
side bands 30, which are fastened to both side surfaces of the second mask 20 so as to be arranged toward the back of the head of the user, and have elasticity;
an upper band 40, which is fastened to the upper side of the second mask 20 so as to be arranged toward the back of the head of the user by passing over the top of the head of the user, and has elasticity; and
a buckle unit 50 for fastening both side bands 30 to the upper band 40 at the back of the head of the user.

2. The face compression beauty mask according to claim 1,
wherein the upper band 40 has stronger elasticity than the side band 30.

3. The face compression beauty mask according to claim 1,
wherein the first mask 10 includes a mask in which polygonal spaces are densely formed on the contact surface 12, one space is from 0.2 mm to 1 mm in size, and the width between the space and the adjacent space is from 0.2 mm to 1 mm in size.

4. The face compression beauty mask according to claim 1,
wherein the first mask 10 is made of silicone, and the second mask 20 is made of a synthetic resin or a metal.

5. The face compression beauty mask according to claim 1,
wherein a first micro hole 16 is formed on the first mask 10, and a second micro hole 26 is formed on the second mask 20, thereby allowing air to pass through the first micro hole 16 and the second micro hole 26.

6. The face compression beauty mask according to claim 1,
wherein an electrode 18 is formed on the first mask 10, and a fine current is provided to the electrode 18, thereby allowing the fine current to flow to the face of the user.

7. The face compression beauty mask according to claim 1,
wherein a vibrator 60 that generates vibration is further disposed on one side of the second mask 20 to apply fine vibrations to the facial skin of the user while the user is undergoing skin beauty care.
